(19) United States
(12) Patent Application Publication
(10) Pub. No.: US 2005/0142121 A1
(43) Pub. Date: Jun. 30, 2005

(54) System for improving acceptance of tissue grafts made from embryonic stem cells

(76) Inventors: Joseph D. Gold, San Francisco, CA (US); Jane S. Lebkowski, Portola Valley, CA (US)

Correspondence Address: GERON CORPORATION, 230 CONSTITUTION DRIVE, MENLO PARK, CA, 94025, US

(21) Appl. No.: 10/949,411

(22) Filed: Sep. 24, 2004

Related U.S. Application Data
(60) Provisional application No. 60/532,700, filed on Dec. 24, 2003.

(51) Int. Cl. A61K 48/00; A61K 31/519

(52) U.S. Cl. 424/93.21; 514/262.1; 514/12

(57) ABSTRACT

This disclosure provides a system for improving survival of cells obtained by differentiating human embryonic stem cells, upon transplantation into a subject for regenerative medicine. Transplanted cells that don't survive normally send out signals that exacerbate the rejection response of the host, which in turn leads to further cell destruction. This invention helps allografts survive and benefit the subject by blocking this signal pathway.

### PRIORITY

This application claims the priority benefit of U.S. Provisional Application 60/532,700 (Geron Docket 137/001), filed Dec. 24, 2003. The priority application is hereby incorporated herein by reference in its entirety.

### BACKGROUND

One of the brave hopes for the future of medical therapy is the concept that cultured human cells will be formulated as pharmaceutical agents for the treatment of serious injury and disease. Organ failure or trauma will be medicated using cells capable of supplementing lost metabolic function. In this way, conditions such as heart disease, spinal cord injury, Parkinson's disease, liver failure, diabetes, and bone degeneration can be treated not just to manage the symptoms, but also to regenerate tissue and reverse the functional deficit.

Specialized cell populations now being grown in culture include neural cells (U.S. Pat. Nos. 5,851,832 and 6,040,180); hepatocyte precursors (U.S. Pat. No. 6,146,889); cardiomyocytes (U.S. Pat. No. 6,491,912) and pancreatic islet cells (U.S. Pat. No. 6,365,385). Cell populations suitable for regenerative medicine can also be obtained by starting with pluripotent stem cells (U.S. Pat. Nos. 6,200,806 and 6,090,622), and then differentiating them into the desired cell type using established protocols (U.S. Pat. No. 6,458,589; WO 01/88104).

Off-the-shelf cellular pharmaceuticals prepared according to some of these methods will typically bear histocompatibility markers of the original host, and have allogeneic differences from the subject intended for treatment. Unless transplanted into an immunologically privileged site, the engrafted cells may be subject to a typical host-mediated immune response leading to rejection of the graft. The attending clinician would then be faced with the necessity of managing the response using anti-rejection drugs, unless the immunological or inflammatory response to the graft tissue can be controlled in another fashion.

Several proposals have been made for preventing rejection of allograft cells given in the context of regenerative medicine. U.S. Pat. No. 5,843,425 (Sachs et al.) explains how T cells present in a tolerizing hematopoietic cell preparation can be depleted using specific antibody. International patent disclosure WO 99/39727 (Sytes et al.) advocates administering the hematopoietic cells in combination with something that inhibits CD40 from interacting with its ligand. According to U.S. Pat. No. 5,876,708 (Sachs et al.), the tolerizing effect of the hematopoietic cells can be supplemented by inactivating T cells in the recipient (e.g., using anti-CD4 or anti-CD8), and administering an immunosuppressive agent (such as cyclosporin A). U.S. Pat. No. 5,858,963, U.S. Pat. No. 5,863,528, and WO 97/41863 (Sachs et al.) outline how tolerance can be induced in an animal model using bone marrow cells in combination with cytokines such as stem cell factor, IL-3, GM-CSF, and IL-10. WO 93/09815 (Sachs et al.) proposes transfecting bone marrow hematopoietic cells with nucleic acid encoding MHC antigen to confer tolerance to a transplanted tissue in a recipient animal.

WO 95/03062 (CellPro) suggests tolerizing a recipient for solid organ transplantation by harvesting cells from the organ donor, enriching for hematopoietic cells (such as CD +ve cells), and infusing them into the recipient before transplant. WO 00/59538 (Johns Hopkins) proposes a method of reducing a specific immune response by administering nucleic acids encoding an apoptosis agent like Fas Ligand.

WO 99/51275 (Osiris Therapeutics) proposes to use mesenchymal stem cells presenting membrane-bound antigen to induce specific T cell anergy, thereby inducing immunosuppression. WO 93/13785 (Sachs et al.), WO 95/21527 (Sachs et al.), WO 97/41863 (Sytes et al.), and U.S. Pat. No. 6,006,752 (Sytes et al.) propose methods for inducing immunotolerance, in which hematopoietic stem cells of a donor animal of one species are administered to a recipient of a second species. This forms mixed chimerism in the recipient, which allows them to receive a graft from the first species.

WO 02/44343 (Geron Corp.) provides a system for promoting graft acceptance, using a toleragenic cell population differentiated from human embryonic stem (hES) cells. U.S. Pat. No. 6,280,718 (Kaufman et al.) and WO 03/050251 (Geron) provide recipes for deriving cells of the hematopoietic lineage from hES cells. U.S. patent application Ser. No. 10/404,770 (Geron) provides a strategy using irradiated hES cells in the undifferentiated form in order to induce tolerance against matched or third-party HLA allotypes.

Although potentially efficacious, the preceding strategies require manufacture, validation, and distribution of two different cell populations: one to treat the patient's condition, and the second cell population to induce immunotolerance and promote acceptance of the first. The invention described in this disclosure provides a new system for improving graft survival that does not require administration of a separate cell population just to induce tolerance.

### SUMMARY OF THE INVENTION

This disclosure is directed towards optimization of the use of pluripotent stem cells—exemplified by human embryonic stem (hES) cells and their equivalents—in regenerative medicine. Specifically, it provides a system for improving the durability of hES derived cells upon transplantation in an allogeneic host. In part, the improved survival comes by preventing transplanted cells that don't survive from sending out signals that exacerbate the inflammatory or immune reaction of the host, which in turn can lead to further cell destruction.

An embodiment of this invention involves preparing cells for engraftment into a subject who would benefit from regenerative medicine using a cell population adapted to decrease the concentration of factors made by the cells if they die following transplant into the subject. Exemplary source of the therapeutic cell population is hES cells (or other pPS cells), differentiated into cells of a certain tissue type, as defined and exemplified below. The cells are then adapted to decrease the concentration of factors that exacerbate rejection, such as uric acid.

The concentration of the exacerbating factor may be decreased by providing a means for reducing the rate of synthesis of the factor by the transplanted cells, or by providing a means for increasing the rate that the factor is removed from around the site of the transplant. One way to affect the local concentration of uric acid is to adapt the cells by treating them with an inhibitor of xanthine oxidase, as exemplified in the sections that follow, after differentiation and before transplantation into the subject. Another way is to genetically alter the cells (either before or after differentiation) using a gene or vector, such that they have reduced ability to synthesize uric acid, or increased ability to remove it. For example, they can be caused to express an enzyme that metabolizes uric acid, or a precursor in the uric acid pathway.

Alternatively or in addition, the differentiated cells may also be prepared by treating them to decrease apoptosis upon transplant into the subject. For example, they can be treated in such a way that the akt kinase pathway is activated, by culturing with erythropoietin, or by any other manipulation that has a similar effect.

Another embodiment of the invention involves preparing a human or non-human mammal to receive a graft of hES or pPS derived cells by administering a medicament comprising a substance that decreases the amount of exacerbating factor produced by the cells after they are engrafted. For reducing local uric acid concentration, suitable substances include inhibitors of xanthine oxidase, enzymes that metabolize uric acid or a precursor in the uric acid pathway, and vectors that encode such enzymes.

The skilled reader has the option of preparing either the allograft cells or the intended recipient in accordance with these embodiments, or both. The clinician then administers the prepared cells to the prepared subject, so as to benefit from the combined effect.

Other embodiments of the invention are cell preparations and other products suitable for carrying out the described methods. One such embodiment is a population of an hES or pPS derived population of differentiated cells that have been adapted to reduce production of uric acid and other exacerbating factors in accordance with the invention. The differentiated cells can be part of a system for the manufacture of pharmaceutical compounds, which may further comprise the undifferentiated hES or pPS cell line from which they were derived. Another such embodiment is a kit comprising hES or pPS derived cells, in combination with a medicament that contains one or more substances for pretreatment of the intended recipient so as to prepare them to receive the allograft in a manner that reduces production of exacerbating factors, in accordance with this invention.

Other embodiments of the invention will be apparent from the description that follows.

### DETAILED DESCRIPTION

The makers of this invention propose that survival of pPS derived cardiomyocyte grafts can be improved by suppressing danger signals that typically triggers the process of rejection. In particular, it is a theory of this invention that uric acid and other factors made by graft cells that do not survive the transplant exacerbate a rejection response. This can stimulate inflammatory or immune meditated tissue destruction, causing the lysis of more cells—leading to a snowball effect that may prevent any of the engrafted cells from surviving.

This disclosure shows how decreasing the amount of uric acid in or around the engrafted tissue can improve graft survival. Decrease in the concentration of uric acid can be effected by inhibiting the transplanted cells from producing it, or by providing a mechanism (in the transplanted cells, or the host, or both) that enables the uric acid to be removed more quickly. Uric acid accumulation from transplanted cells can be decreased either by pretreating the cells with allopurinol which inhibits xanthine oxidase, by pretreating the intended recipient with allopurinol or uricase (which diminishes endogenous uric acid), or by pretreating both the cells and the recipient in this way. This in turn prevents exacerbation of the rejection response directed against the engrafted tissue, which improves survival and leads to extended function of the graft.

Decreasing the amount of uric acid present can be combined with a treatment that diminishes the extent of apoptosis in the graft. For example, heat shock, culturing with erythropoietin, pretreatment with anti-inflammatory agents, or a combination of these manipulations can greatly enhance survival of engrafted pPS derived cells. By treating the cells both to decrease apoptosis and limit uric acid production, the number of cells that initially survive the transplant is increased, and the immune effects of the non-surviving cells is minimized.

Definitions

Prototype “primate Pluripotent Stem cells” (pPS cells) are pluripotent cells derived from pre-embryonic, embryonic, or fetal tissue at any time after fertilization, and have the characteristic of being capable under appropriate conditions of producing progeny of several different cell types that are derivatives of all of the three germinal layers (endoderm, mesoderm, and ectoderm), according to a standard art-accepted test, such as the ability to form a teratoma in 8-12 week old SCID mice. The term includes both established lines of stem cells of various kinds, and cells obtained from primary tissue that are pluripotent in the manner described.

pPS cell cultures are described as “undifferentiated” when a substantial proportion of stem cells and their derivatives in the population display morphological characteristics of undifferentiated cells, clearly distinguishing them from differentiated cells of embryo or adult origin.

Cell populations described as over 75%, 90%, or 98% homogeneous for cells of one tissue type contain cells that are typical of that organ or tissue type as a stated minimum percentage. It is recognized that cell populations either in vivo or in tissue culture vary someone in phenotype, comprising cells bearing different markers and having different functions. For example, skin may contain epithelial cells, fibroblasts, and endothelial cells. Nevertheless, if taken from a single tissue, the population will typically still be homogeneous according to this definition.

The term “uric acid” means not only the acid form of the compound (C₅H₄N₄O₃), but also the conjugate base, salts thereof, and any chemically equivalent form that may be present in a biological environment such as an allograft transplant site.

General References

General methods in cell biology, protein chemistry, and antibody techniques can be found in Current Protocols in Protein Science (J. E. Colligan et al. eds., Wiley & Sons); Current Protocols in Cell Biology (J. S. Bonifacino et al., Wiley & Sons) and Current protocols in Immunology (J. E. Colligan et al. eds., Wiley & Sons.). Reagents, cloning vectors, and kits for genetic manipulation referred to in this disclosure are available from commercial vendors such as BioRad, Stratagene, Invitrogen, ClonTech, and Sigma-Aldrich Co.

Cell culture methods are described generally in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Tissue culture supplies and reagents are available from commercial vendors such as Gibco/BRL, Nalgene-Nunc International, Sigma Chemical Co., and ICN Biomedicals. Other references include Allopurinol: A Medical Dictionary, Bibliography, and Annotated Research Guide (ICON Health Publications), and How do you Solve a Problem Like Urea (R. Rogers & O. Hammerstein II, RCA).

Sources of Stem Cells

This invention can be practiced using stem cells of various types. Particularly suitable for use in this invention are primate pluripotent stem (pPS) cells derived from tissue formed after gestation, such as a blastocyst, or fetal or embryonic tissue taken any time during gestation. Non-limiting examples are primary cultures or established lines of embryonic stem cells or embryonic germ cells, as described below. The techniques of this invention can also be implemented directly with primary tissue, deriving differentiated cells such as neural cells directly from early embryonic cells without first establishing an undifferentiated cell line, or harvesting committed progenitors from neural tissue or other samples obtained from fetal or adult material.

Embryonic Stem Cells

Embryonic stem cells can be isolated from blastocysts of primate species (U.S. Pat. No. 5,843,780; Thomson et al., Proc. Natl. Acad. Sci. USA 92:7844, 1995). Human embryonic stem (hES) cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hES cells include their pluripotent derivatives, such as primitive ectoderm-like (EPL) cells, outlined in WO 01/51610 (Bresagen).

hES cells can be obtained from human preimplantation embryos (Thomson et al., Science 282:1145, 1998). Alternatively, in vitro fertilized (IVF) embryos can be used, or one-cell human embryos can be expanded to the blastocyst stage (Bongso et al., Hum Reprod 4: 706, 1989). The zona pellucida of the blastocyst is removed, and the inner cell masses are isolated. The intact inner cell mass can be plated on mEF feeder layers, and after 9 to 15 days, inner cell mass derived outgrowths are dissociated into clumps, and replated. ES-like morphology is characterized as compact colonies with apparently high nucleus to cytoplasm ratio and prominent nucleoli. Resulting ES cells are then routinely split every 1-2 weeks.

hPS cells can be propagated continuously in culture, using culture conditions that promote proliferation while inhibiting differentiation. Traditionally, ES cells are cultured on a layer of feeder cells, typically fibroblasts derived from embryonic or fetal tissue (Thomson et al., Science 282:1145, 1998).

Scientists at Geron have discovered that hPS cells can be maintained in an undifferentiated state even without feeder cells. The environment for feeder-free cultures includes a suitable culture substrate, such as Matrigel® or laminin. The cultures are supported by a nutrient medium containing factors that promote proliferation of the cells without differentiation (WO 99/20741). Such factors may be introduced into the medium by culturing the medium with cells secreting such factors, such as irradiated primary mouse embryonic fibroblasts, telomerized mouse fibroblasts, or fibroblast-like cells derived from hPS cells (U.S. Pat. No. 6,642,048). Medium can be conditioned by plating the feeders in a serum free medium such as Knock-Out DMEM (Gibco), supplemented with 20% serum replacement (U.S. 2002/0076747 A1, Life Technologies Inc.) and 4 ng/mL bFGF. Medium that has been conditioned for 1-2 days is supplemented with further bFGF, and used to support hPS cell culture for 1-2 days (WO 01/51616; Xu et al., Nat. Biotechnol. 19:971, 2001).

Alternatively, fresh non-conditioned medium can be used, if supplemented with added factors (like a fibroblast growth factor or forskolin) that promote proliferation of the cells in an undifferentiated form. Exemplary is a base medium like X-VIVO™ 10 (Biowhittaker) or OBSF™-60 (Quality Biological Inc.), supplemented with bFGF at 40-80 ng/mL, and optionally containing stem cell factor (15 ng/mL), or Flt3 ligand (75 ng/mL). These medium formulations have the advantage of supporting cell growth at 2-3 times the rate in other culture systems (WO 03/020920).

Under the microscope, ES cells appear with high nuclear/cytoplasmic ratios, prominent nucleoli, and compact colony formation with poorly discernable cell junctions. Primate ES cells typically express the stage-specific embryonic antigens (SSEA) 3 and 4, and markers detectable using antibodies designated Tra-1-60 and Tra-1-81. Undifferentiated hES cells also typically express the transcription factor Oct-3/4, Cripto, gastrin-releasing peptide (GRP) receptor, podocalyxin-like protein (PODXL), and human telomerase reverse transcriptase (hTERT), as detected by RT-PCR (US 2003/0224411 A1).

Other Stem Cells

The illustrations provided in the Example section ensue from work done with human embryonic stem cells. However, except where otherwise specified, the invention can be practiced using multipotent cells of any vertebrate species, including pluripotent stem cells from humans; non-human primates, and other non-human mammals.

By no means does the practice of this invention require that a human blastocyst be disaggregated in order to produce the hPS or embryonic stem cells for practice of this invention. hES cells can be obtained from established lines obtainable from public depositories (for example, the WiCell Research Institute, Madison Wis. U.S.A., or the American Type Culture Collection, Manassas Va., U.S.A.). Human Embryonic Germ (hEG) cells can be prepared from primordial germ cells as described in Shamblott et al., Proc. Natl. Acad. Sci. U.S.A. 95:13726, 1998 and U.S. Pat. No. 6,090,622. U.S. Patent Publication 2003/0113910 A1 reports pluripotent stem cells derived without the use of embryos or fetal tissue. It may also be possible to reprogram other progenitor cells into hPS cells by using a factor that induces the pluripotent phenotype (Chambers et al., Cell 113:643, 2003; Mitsui et al., Cell 113:631, 2003). Under appropriate conditions, any cell with appropriate proliferative and differentiation capacities can be used for the derivation of differentiated tissues for use according to this invention.

Differentiating PPS Cells into Tissue for Transplantation

Differentiated cell preparations for use in transplantation can be made from pPS cells according to established methods.

By way of illustration, neural cells can be generated from pPS cells according to the method described in International Patent Publication WO 01/88104 and WO 03/000868 (Geron Corporation). Undifferentiated pPS cells or embryoid body cells are cultured in a medium containing one or more neurotrophins and one or more mitogens, generating a cell population in which at least ˜60% of the cells express A2B5, polysialylated NCAM, or Nestin and which is capable of at least 20 doublings in culture. Exemplary mitogens are EGF, basic FGF, PDGF, and IGF-1. Exemplary neurotrophins are NT-3 and BDNF. The proliferating cells can then be caused to undergo terminal differentiation by culturing with neurotrophins in the absence of mitogen. Cell populations can be generated that contain a high proportion of cells staining for tyrosine hydroxylase, a characteristic of dopaminergic neurons.

Oligodendrocytes can be generated from pPS cells by culturing them as cell aggregates, suspended in a medium containing a mitogen such as FGF, and oligodendrocyte differentiation factors such as triiodothyronine, selenium, and retinoic acid. The cells are then plated onto a solid surface, the retinoic acid is withdrawn, and the population is expanded. Terminal differentiation can be effected by plating on poly-L-lysine, and removing all growth factors. Populations can be obtained in which over 80% of the cells are positive for oligodendrocyte markers NG2 proteoglycan, A2B5, and PDGFRα, and negative for the neuronal marker NeuN. See PCT publication WO 04/007696 (Keirstead).

Hepatocytes can be generated from pPS cells according to the method described in U.S. Pat. No. 6,458,589 and PCT publication WO 01/81549 (Geron Corporation). Undifferentiated pPS cells are cultured in the presence of an inhibitor of histone deacetylase. In an exemplary method, differentiation is initiated with 1% DMSO (4 days), then 2.5 mM of the histone deacetylase inhibitor n-butyrate. The cells obtained can be matured by culturing 4 days in a hepatocyte culture medium containing n-butyrate, DMSO, plus growth factors such as EGF, hepatocyte growth factor, and TGF-α. Other effective hepatocyte differentiation protocols are described in U.S. Ser. No. 10/810,311.

Cardiomyocytes or cardiomyocyte precursors can be generated from pPS cells according to the method provided in WO 03/006950. The cells are cultured in a growth environment comprising fetal calf serum or serum replacement, and optionally a cardiotrophic factor that affects DNA-methylation, such as 5-azacytidine. Spontaneously contracting cells can then be separated from other cells in the population, by density centrifugation. Further process steps can include culturing the cells so as to form cardiac bodies, removing single cells, and then dispersing and reforming the cardiac bodies in successive iterations, as described in U.S. Ser. No. 10/805,099.

Hematopoietic cells can be made by coculturing pPS cells with murine bone marrow cells or yolk sac endothelial cells was used to generate cells with hematopoietic markers (U.S. Pat. No. 6,280,718). Hematopoietic cells can also be made by culturing pPS cells with hematogenic cytokines and a bone morphogenic protein, as described in U.S. 2003/0153082 A1 and WO 03/050251.

Osteoblasts and their progenitors can be generated from pPS cells according to the method described in WO 03/004605. pPS-derived mesenchymal cells are differentiated in a medium containing an osteogenic factor, such as bone morphogenic protein (particularly BMP-4), a ligand for a human TGF-β, receptor, or a ligand for a human vitamin D receptor. Cells that secrete insulin or other pancreatic hormones can be generated by culturing pPS cells or their derivatives in factors such as activin A, nicotinamide, and other factors listed in WO 03/050249. Chondrocytes or their progenitors can be generated by culturing pPS cells in microaggregates with effective combinations of differentiation factors listed in WO 03/050250.

In principle, any transplanted cells or tissue at risk for rejection will benefit from the immunotolerance strategy described in this application.

Preparing the Cells and the Subject for Transplantation

According to this invention, the allograft cells or the recipient subject, or both, are prepared for transplantation in such a way so as to decrease the production of uric acid, related compounds, and other factors that exacerbate inflammation, immune recognition or rejection of the transplanted tissue.

Improved survival of the allograft tissue can be accomplished by adapting the therapeutic cell population to decrease production of factors such as uric acid, or to increase the rate that uric acid is metabolized or removed, or both. Xanthine oxidase which dying cells use to produce uric acid can be inhibited with compounds such as allopurinol, oxypurinol, and BOF-4272 (Kogler et al., Cardiovasc Res. 59:582, 2003; Naito et al, Biol Pharm Bull. 25:674, 2002; Shi et al., Nature 425:516, 2003). Pre-treatment of the cells with low levels of tungsten would deplete cellular levels of molybdenum, a necessary co-factor for xanthine oxidase (Suzuki et al., Proc. Natl. Acad. Sci. USA 95:4754, 1998), and may also reduce uric acid production.

Another way to reduce xanthine oxidase activity in the cells is to decrease the amount of xanthine oxidase mRNA. Transient inactivation just before administration can be accomplished by treating the cell with mRNA antisense, ribozyme, or siRNA that is complementary or specific for the xanthine oxidase gene sequence. Longer-term activation can be accomplished by inactivating or modifying the gene encoding xanthine oxidase on one or both alleles, or by introducing a transgene encoding RNA antisense, ribozyme, or siRNA. The transgene can be placed under control of a promoter inducible with compounds such as tetracycline (Shockett et al., Proc. Natl. Acad. Sci. USA 92:6522, 1995; Rossi et al., Molec. Cell 6:723, 2000) or heavy metals (Yan et al., Biochim. Biophys. Acta 1679:47, 2004). In this way, the genetic alteration can be done at any stage, allowing xanthine oxidase to be down-regulated just before administration by combining the cells with the inducing compound.

Alternatively or in addition, the allograft tissues can be adapted by causing them to express an enzyme that degrades or causes sequestration of the exacerbating factor. For uric acid, suitable enzymes are uricase, and natural or recombinant forms of urate oxidase (e.g., Rasburicase™, Fasturtec™, Elitek™). A gene sequence encoding the enzyme (or a catalytically active fragment thereof) is used to transfect the cell, either at the stage of the undifferentiated pPS cell, or subsequent to differentiation. The enzyme may be expressed within the cell, or exported so as to create a milieu free of uric acid near the transplant site. Vectors such as lipofectamine conjugates and adenovirus can be used for transient expression, or vectors such as retrovirus, lentivirus, and adeno-associated virus can be used in situations where long-term expression by the cell and its progeny is more desirable. Methods and reagents for producing genetically altered pPS cells and their progeny are described in U.S. Ser. No. 09/849,022, which is hereby incorporated herein by reference.

A further adaptation of the allograft cells before transplant can be done to minimize the extent of initial cell death, which otherwise provides the initial burst of uric acid production. One means of making the cells less subject to apoptosis is to activate Akt kinase activity (Matsui et al., Circulation 104:330, 2001). This can be done, for example, by culturing with growth factors such as erythropoietin (EPO), insulin, and IGF-1, and gp130 activators such as IL-6, cardiotropin, IL-11, and CNTF. Another way of activating Atk kinase is by heat shock: raising the temperature of the cells by about 6° C. above normal culture temperatures for 15 min to 2 h at a suitable period (say, 1 or 3 days) prior to use for transplantation or preparation of the medicament. Alternatively or in addition, the cells can be treated with a non-steroidal anti-inflammatory agent such as ibuprophen.

The subject to receive the transplant can also be adapted to reduce exacerbating factors from accumulating in the milieu of the allograft. For uric acid, the subject can be treated locally or systemically with one or more of the aforementioned inhibitors of xanthine oxidase. They can also be treated locally or systemically with one or more of the enzymes and other substances that metabolize or sequester uric acid, such as uricase or urate oxidase. Local treatment with a vector causing transient expression of uricase or urate oxidase is also contemplated, preceding, concurrently, or shortly following implantation of the allograft tissue.

Once the cells or the transplant subject, or both, have been adapted as described, the allograft can then be put in place by a suitable procedure for administration of cells to the target site. The use of the materials of this invention in accordance with standard surgical methods is the responsibility of the treating clinician. Following treatment, patients are monitored for general health, survival of the allograft cells, and recovery of physiological function associated with the transplant tissue.

The prepared cell population is typically supplied in the form of a pharmaceutical composition, comprising an isotonic excipient prepared under sufficiently sterile conditions for human administration. Effective cell and medicine combinations can be packaged and distributed separately, or in separate containers in kit form, or (for simultaneous administration to the same site) they can be mixed together.

This invention also includes reagent systems for the production of differentiated cells to be used with this invention. An example is a set or combination of cells that exist at any time during manufacture, distribution, or use of the differentiated cell populations, comprising any combination of two or more cell populations described in this disclosure, such as the differentiated cell population used for therapy, in combination with undifferentiated pPS cells from which they were derived.

For general principles in formulating cell compositions, the reader is referred to Cell Therapy: Stem Cell Transplantation, Gene Therapy, and Cellular Immunotherapy (G. Morstyn & W. Sheridan eds., Cambridge University Press, 1996). Compositions and combinations intended for pharmacological distribution and use are optionally packaged with written instructions for a desired purpose, such as the regeneration of tissue function, genetic therapy, or induction of immune tolerance.

The example that follows is provided by way of further illustration, and is not meant to limit the claimed invention.

### EXAMPLE

Experiments conducted elsewhere have shown that the vast majority of engrafted cardiomyocytes derived from neonatal or adult animals, die within 1-7 days after transplant into the hearts of recipient animals. A significant fraction of cell death is attributed to apoptosis, and the inclusion of a heat shock treatment to the cells before transplant confers significant protection from apoptosis (Zhang et al., J Mol Cell Cardiol. 33:907, 2001). Under some conditions, when hES-derived cardiomyocytes are transplanted into the hearts of acutely infarcted SCID/bg mice, only a small percentage of cells survive.

The makers of this invention have discovered that cardiac improvement upon grafting with hES derived cardiomyocytes (measured by echocardiography) is considerably enhanced when the grafts are pre-treated with erythropoietin, ibuprophen, allopurinol, or a combination of these agents, followed by transplantation into animals optionally pre-conditioned with allopurinol and uricase.

Cardiomyocytes were differentiated from human embryonic stem (hES) cells and purified by density gradient centrifugation on Percoll™, according to established methods (WO 03/006950). They were adapted to decrease uric acid production upon engraftment by culturing for 24 hours in standard differentiation medium (containing 20% FBS) to which had been added 50 μg/mL allopurinol, or 0.5 Units/mL human recombinant erythropoietin (EPO). Alternatively, the cells were heat shocked in standard differentiation medium (20% FBS) by incubating at 43° C. for 45 minutes, and then transferring to a 37° C. incubator 24 h prior to harvest.

Recipient mice were prepared to decrease uric acid production by the engrafted cells by daily injection of 800 μg allopurinol and/or 10 μg uricase intramuscularly for each of the 3 days before transplant.

The transplant experiments were performed using a mouse model for coronary infarction in an external research laboratory under a Research Agreement with Geron Corporation. The left anterior descending artery was ligated as follows. Mice were anesthetized in an isoflurane inhalation chamber and received intraperitoneal injection of Ketanest/Xylazine (50 mg/kg). They were then intubated and ventilated for the entire surgical procedure. Rectal temperature was maintained at 37° C. by a thermostatically regulated heating pad. One ligation with a 9.0 silk stitch was performed on the proximal 2 mm portion of the artery. A pale area demarcated on the surface of the left ventricle resulted in significant left ventricular ischemia encompassing the middle and apical portion of the ventricle.

Using a 25 g needle, 0.25-2×10⁶ cells (suspended in differentiation medium without serum) were injected into the demarcated area. Immediately afterwards, a chest tube (16 G Angio-cath) was inserted and the chest was closed in layers. Ventilation was maintained until there was sufficient spontaneous breathing, and extubation followed. The ventricular diameter was measured after three weeks by echocardiography. Fractional shortening (a measure of ventricular diameter)≡[(diameter at diastole minus diameter at systole)÷(diameter at diastole)]. Results are shown in Table 1.

TABLE 1

| | Fractional |
|---|---|
| | shortening |
| | at 3 weeks |
| Treatment Group | post-treatment |
| Noninfarcted animals | 25-30% |
| Infarcted | 13% |
| Infarcted + injected human fibroblasts | 17% |
| Infarcted + injected hES-derived cardiac preparation | 19% |
| Infarcted + injected hES-derived cardiac preparation + | 19% |
| heat shock | |
| Infarcted + injected hES-derived cardiac preparation + | 19% |
| heat shock (cells) | |
| Infarcted + injected hES-derived cardiac preparation + | 22% |
| ibuprofen (animals) | |
| Infarcted + injected hES-derived cardiac preparation + | 22.4% |
| EPO (cells) | |
| Infarcted + injected hES-derived cardiac preparation + | 24% |
| allopurinol (cells and animals) | |

Reduction of uric acid by adapting the cardiomyocytes or recipient animals with allopurinol correlates with an increased functional improvement (24%) compared with the control (13%), or animals transplanted with cardiomyocytes without any adaptation (19%). This is consistent with improved acceptance of the transplanted cells by the host, due to decreased uric acid production leading to a less severe rejection response.

Pretreating the cells with EPO or heat shock also correlated with improved function. EPO protects cells in both the brain and the heart from ischemia-induced death by activation of the akt kinase pathway, the same pathway that is activated in some cells by heat shock. The inventors hypothesize that the beneficial effects of EPO shown in Table 1 are due to its anti-apoptotic effects on the hES-derived cells. The lower fractional shortening observed after engraftment of heat shocked hES-derived grafts may be due to either less efficient induction of protective effects, or the fact that heat shock actually stimulates apoptosis in some cell types.

The compositions and procedures described in this disclosure can be effectively modified by routine optimization without departing from the spirit of the invention embodied in the claims that follow.

1. A method of regenerative medicine, comprising preparing cells for engraftment by differentiating human embryonic stem cells into a particular tissue type, and adapting the cell population to decrease the amount of uric acid produced by the cells upon transplantation into a subject.

2. The method of claim 1, wherein the cells are adapted by treating them with an inhibitor of xanthine oxidase after differentiation and before transplantation into the subject.

3. The method of claim 2, wherein the inhibitor is allopurinol.

4. The method of claim 2, wherein the inhibitor is oxypurinol or BOF-4272.

5. The method of claim 2, wherein the inhibitor is a xanthine oxidase mRNA antisense molecule, ribozyme, or siRNA.

6. The method of claim 1, wherein the cells are adapted by genetically altering them (either before or after differentiation) to express an enzyme that metabolizes uric acid.

7. The method of claim 6, wherein the enzyme is uricase.

8. The method of claim 6, wherein the enzyme is a urate oxidase.

9. The method of claim 1, further comprising treating the differentiated cells so as to decrease apoptosis of the cells upon transplant into the subject.

10. The method of claim 9, wherein the cells are treated by culturing them with erythropoietin.

11. The method of claim 1, wherein the cell population is a population of neurons, oligodendrocytes, cardiomyocytes, hepatocytes, islet cells, hematopoietic cells, mesenchymal cells, osteoblasts, or chondrocytes.

12. A method of regenerative medicine, comprising preparing a subject to receive a graft of cells differentiated from human embryonic stem cells by administering to the subject a substance that decreases the amount of uric acid produced by the cells after they are engrafted.

13. The method of claim 12, wherein the subject is administered an inhibitor of xanthine oxidase.

14. The method of claim 12, wherein the inhibitor is selected from allopurinol, oxypurinol and BOF-4272.

15. The method of claim 12, wherein the subject is administered an enzyme that metabolizes uric acid.

16. The method of claim 15, wherein the enzyme is uricase or a urate oxidase.

17. The method of claim 12, wherein the subject is administered both an inhibitor of xanthine oxidase, and an enzyme that metabolizes uric acid.

18. A method of regenerative medicine, comprising preparing cells for engraftment into a subject according to claim 1, and also preparing the subject to receive the prepared cells according to the method of claim 12.

19. A method of regenerative medicine, comprising administering to a subject prepared according to claim 12 with cells prepared according to the method of claim 1.

20. A cell population differentiated from human embryonic stem cells and adapted for regenerative medicine according to the method of claim 1.

21. The cell population of claim 20, which is a population of neurons, oligodendrocytes, cardiomyocytes, hepatocytes, islet cells, hematopoietic cells, mesenchymal cells, osteoblasts, or chondrocytes.

22. A kit for use in regenerative medicine, comprising the adapted cell population of claim 20, in combination with one or more substances to prepare a subject to be transplanted with the adapted cell population, selected from inhibitors of xanthine oxidase and enzymes that metabolize uric acid.
